(19) United States
(12) Patent Application Publication
(10) Pub. No.: US 2020/0268531 A1
(43) Pub. Date: Aug. 27, 2020

(54) PROSTHESIS FOR A LOWER EXTREMITY AND A CONNECTION DEVICE FOR SUCH AN EXTREMITY

(71) Applicant: Ottobock SE & Co. KGaA, Duderstadt (DE)

(72) Inventor: Hannes KRENZ, Dresden (DE)

(73) Assignee: Ottobock SE & Co. KGaA, Duderstadt (DE)

(21) Appl. No.: 16/642,341

(22) PCT Filed: Sep. 4, 2018
(86) PCT No.: PCT/EP2018/073749

§ 371 Date: Feb. 26, 2020

(30) Foreign Application Priority Data
Sep. 4, 2017 (DE) 10 2017 120 257.9

(51) Int. Cl. A61F 2/66; A61F 2/64; A61F 2/78; A61F 2/76

(52) U.S. Cl. CPC A61F 2/6607; A61F 2/64; A61F 2002/607; A61F 2/76; A61F 2/78

(57) ABSTRACT

A prosthesis for a lower extremity, the prosthesis including a first prosthesis component and a second prosthesis component, wherein the first prosthesis component can be locked relative to the second prosthesis component by way of at least one locking element, wherein the prosthesis comprises at least one holding device, by way of which the first prosthesis component is detachably held on the second prosthesis component.

The invention relates to a prosthesis for a lower extremity, wherein the prosthesis comprises a first prosthesis component and a second prosthesis component, wherein the first prosthesis component can be locked relative to the second prosthesis component by way of at least one locking element. The invention also relates to a connection device for such a prosthesis.

Such prostheses are described in U.S. Pat. No. 5,326,352, for example.

For wearers of a lower extremity prosthesis, such as a leg prosthesis or a foot prosthesis, it is practical in various situations to swap certain components of the prosthesis: for example, swapping a prosthetic foot that is well-suited to normal walking and standing for a sports foot when the patient wishes to engage in sporting activities. In other cases, it may be practical to completely remove one or several prosthesis components which, for instance, on long car journeys are not used and simply a nuisance. Therefore, the prior art has tried for many years to make it possible to detach different prosthesis components from one another as quickly and easily as possible, and to re-attach them to one another. For instance, US 2003/0650647 describes a prosthesis adapter with a tubular prosthetic element that is inserted into a bracket, the circumference or cross-section of which can be enlarged or reduced by a clamping device. To remove the tubular prosthetic element, the bracket is expanded and the prosthesis component can be removed. If it is to be re-assembled, it is inserted into the expanded bracket; the cross-section of the bracket is then reduced via the clamping device, which may be a bracket tension lever for instance, and the prosthesis component thus clamped in. However, it is impractical that a precise positioning of the tubular prosthetic element, especially in the axial direction, and an angular adjustment of the two prosthesis components to be connected cannot be adjusted in a way that is reproducible. The corresponding adjustment information is lost upon the removal of the prosthesis components. U.S. Pat. No. 9,693,884 B1 contains a different prosthesis adapter.

The instruction in U.S. Pat. No. 5,326,352 therefore offers the advantage that the two components of the prosthesis can only be fixed in certain angular orientations to each other and only at a distance from each other. To this end, the prosthesis features a projection on one prosthesis component and a corresponding recess in the other prosthesis component. If these elements are arranged in relation to one another in such a way that the projection is inserted into the recess, both the relative distance of the two components and their angular orientation to each other are fixed. In this case, bores provided in the projection and the respective other prosthesis component are moved so that they overlap with one another, such that said bores are locked in relation to one another with a pin, which in this case forms the locking element.

The locking of the two prosthesis components in relation to one another means that a movement of the two components relative to one another is no longer possible. This does not refer to movements which occur due to a play between the prosthesis components or due to production tolerances.

However, it is impractical that the two components have to be held or otherwise fixed when being locked in the respective locking position which enables a locking. This may be achieved, for example, by subjecting the prosthesis to a load. However, this is complex and not always easy to achieve, particularly for motor-impaired persons, and may cause the wearer of the prosthesis to feel a sense of insecurity. The invention thus aims to further develop a prosthesis according to the generic term in claim 1 so that it is safe and easy to use.

The invention solves the problem by way of a prosthesis according to the generic term in claim 1, characterized in that the prosthesis comprises at least one holding device, by way of which the first prosthesis component is detachably held on the second prosthesis component.

If the prosthesis for the lower extremity, for example a leg or foot prosthesis, is in use, the two prosthesis components are connected to one another. The locking element has locked the two prosthesis components in relation to one another such that a movement of the two components away from one another is not possible. Here, the locking element may be a pin, a clip or a screw, for example. It may be designed to be a separate component or as a single piece with the first prosthesis component or the second prosthesis component. Of course, other locking elements are also possible. If the first prosthesis component is now removed from the second prosthesis component, for instance because an artificial foot is to be replaced by another foot, the locking element is first detached, for example removed, as is known from the prior art. The lock is thus released. However, due to the holding device provided according to invention, the two prosthesis components are still held together so that an inadvertent release of the locking element, for instance, does not cause an unintended removal of the second prosthesis component from the first prosthesis component. It is therefore not possible, for example, for a prosthetic foot to fall away from the rest of the prosthesis following the release of the locking element. Given that the holding device holds the two prosthesis components together such that they can be detached from one another, the two prosthesis components can be easily removed from one another, for example by overcoming a holding force which—in a preferred configuration—is applied by the holding device.

The force applied by the holding device, which holds the two prosthesis components together even when they are not locked, is preferably great enough to ensure that the two prosthesis components do not fall away from one another if they are exposed to gravity. The force does not have to be so great that the prosthesis components are held together even when the prosthesis is in use, for example in a swing phase of a step, if they are not locked. For example, the force applied by the holding device is at least 10 N, preferably at least 15 N, especially preferably at least 20 N. A force of this size is enough to hold a prosthetic foot with a normal shoe.

With regards to the assembly of two prosthesis components of a prosthesis according to the invention, the two prosthesis components are arranged relative to one another such that that they are already held by the holding device. In this state, the two components can no longer be separated from one another by the acting gravitational force, so that a prosthesis component in use is prevented from falling off the rest of the prosthesis. In this secure state, the wearer of the prosthesis has both hands free to use the locking element and lock the two prosthesis components relative to one another; this ensures operational safety, including when the prosthesis is in use.

In a preferred configuration, the holding device has a first holding element, which is arranged on the first prosthesis component, and a second holding element, which is arranged on the second prosthesis component. The two holding elements may be provided as part of a separate connection device, so that existing prostheses can also be modified and benefit from the advantages of the invention. Preferably, the first holding element and the second holding element are positive-locking elements, in particular velcro elements, which are designed to correspond to one another. When it comes to assembly, the two prosthesis components are placed against each other in such a way that, in this case, the positive-locking elements are engaged with one another; as a result, a detachment of the two prosthesis components from one another is only possible by overcoming the corresponding holding force, for example in the case of velcro or push-button elements, or by activating a corresponding unlocking device, for example by displacing a snap element. However, the engagement and detachment is easily achieved, preferably using just one hand. Even for persons with limited motor skills, the engagement of the various positive-locking elements can be achieved in an easy and secure fashion. As a result, a holding force is applied which prevents an accidental loss of one or both of the prosthesis parts; however, said holding force is preferably not sufficient to ensure operational safety of the prosthesis. This only happens when, for example, the positive-locking elements are already engaged with each other, the locking element is used and the two prosthesis components are locked together in this state, in which the holding device is already effective.

Alternatively or additionally, the first holding element may also be able to exert a holding force, especially a magnetic holding force, on the second holding element. To this end, it has been proven practical for the first holding element and/or the second holding element to feature at least one, but preferably several, permanent magnets. If both holding elements each comprise a permanent magnet, the orientation of the magnets must of course be selected in such a way that when the two prosthesis components are arranged next to one another, opposite poles of the two permanent magnets are arranged towards each other so that they attract each other, thereby resulting in a holding force between the two holding elements. In a structurally simple configuration, only the first holding element or the second holding element is equipped with at least one, but preferably several, magnets. In this case, the respective other holding element comprises at least one magnetizable element, so that a holding force between the two holding elements can occur. This is especially beneficial if the prosthesis component to be detached, such as a prosthetic foot that is to be removed from the rest of an attached leg prosthesis or a leg prosthesis that is to be removed from an applied socket, is not equipped with a magnet. In this case, unwanted forces of attraction between the removed prosthesis component and other objects, for example metallic objects, can be prevented from occurring.

The first holding element is preferably arranged on a first contact element and the second holding element on a second contact element which lie next to one another when the first prosthesis component is locked relative to the second prosthesis component. Here, the first contact element preferably features at least one projection and the second contact element preferably features a recess that is designed to correspond to the projection. This has the advantage that, for instance, an orientation of the two prosthesis components when they are placed against each other and particularly when they are locked is fixed and therefore adjustable in a manner that can be reproduced. The components may also be designed to self-orienting in relation to one another which, as the expert knows from the prior art, can be achieved by certain shapes of the projection and/or the recess. This renders the prosthesis easier to handle, thereby further increasing its acceptance by the patient.

Preferably, the holding device can be released with one hand and preferably without a tool; it is practical if this requires a movement in just one direction. Such a movement refers, for instance, to a movement that can be performed with one hand without having to “re-grasp” the prosthesis component, i.e. without having to release the respective prosthesis component and re-grasp it in a different configuration. It may be a pulling movement in one direction, which does necessarily have to be conducted in an exactly straight line; rather, it may be a curved movement.

The prosthesis components can preferably only be locked when the holding device holds the prosthesis components against one another and/or the holding device can only be released when the prosthesis components are unlocked. This determines an order in which the individual steps must be carried out. When connecting the prosthesis components, the holding device is first activated by arranging the holding elements next to one another, such that the force is exerted. This preferably leads to a positioning of the prosthesis components in relation to one another that enables the locking device, along with the locking element, to be activated and locked. When detaching the two components from one another, both components must first be unlocked before the force applied by the holding device can be overcome and the two prosthesis components separated.

The invention also solves the problem by way of a connection device for a prosthesis, wherein the connection device comprises a holding device with a first holding element for arranging on a first prosthesis component and a second holding element for arranging on a second prosthesis component, and the locking element. The holding device is configured to apply the corresponding holding force. Both holding elements may be connected to the respective prosthesis component, for example, via prefabricated adapters, arranged connection elements or in another way, such that even conventional prosthesis components can benefit from the advantages of the present invention.

On the one hand, if such a connection device is used in the prosthesis components to be connected, the weight of the prosthesis can be reduced, preferably by several 100 g per connection device used. On the other hand, the required installation space and in particular the installation height is reduced by 40 mm to 80 mm, for example, depending on the prosthesis component, by foregoing the distal and proximal connection pieces. This is especially practical for patients with a long amputation stump, such as a long upper leg stump or lower leg stump, who cannot use conventional connection devices due to the required installation height. The integration of the connection device into the components means that this is no longer the case.

Thanks to the prostheses and connection devices described here, different prosthesis components can be quickly, easily and securely connected to one another. For example, this renders it possible to quickly swap an everyday prosthesis for a sports prosthesis, such as a swimming prosthesis. This is not restricted to individual components, such as artificial feet or knees: even entire prosthetics comprising several components can be exchanged. In this way, an entire upper leg prosthesis may be exchanged, wherein only the prosthesis socket remains on the amputation stump.

In the following, an example of an embodiment of the present invention will be explained in more detail by way of the attached figures: They show:

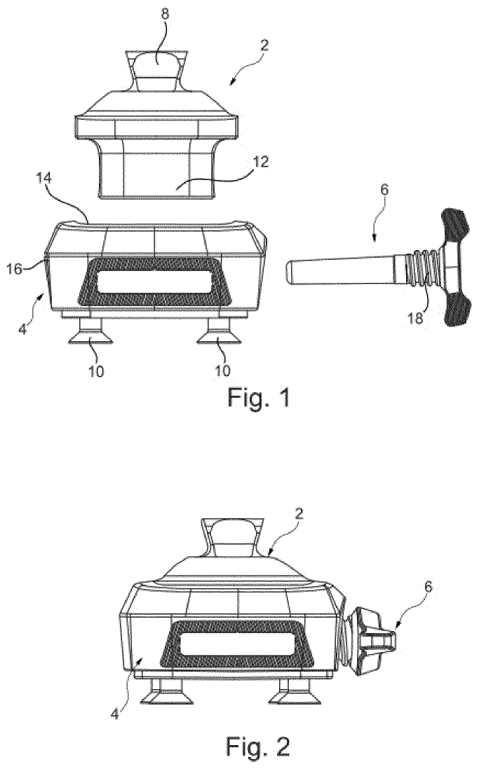

FIG. 1—a connection device according to a first example of an embodiment of the present invention when in the unconnected state,

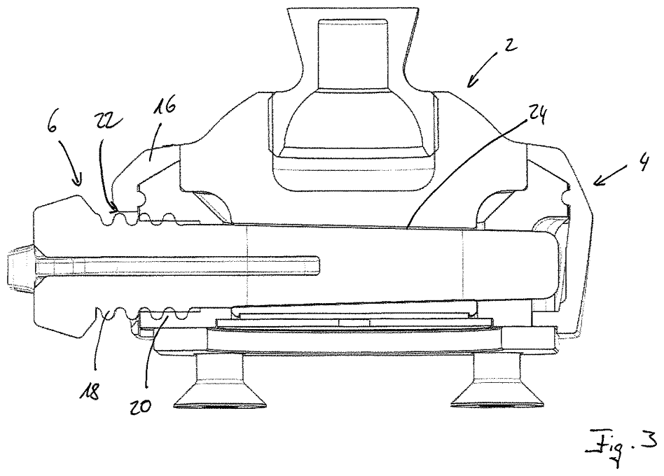

FIG. 2—the connection device from FIG. 1 in the connected state,

FIG. 3—the connection device in a first sectional view,

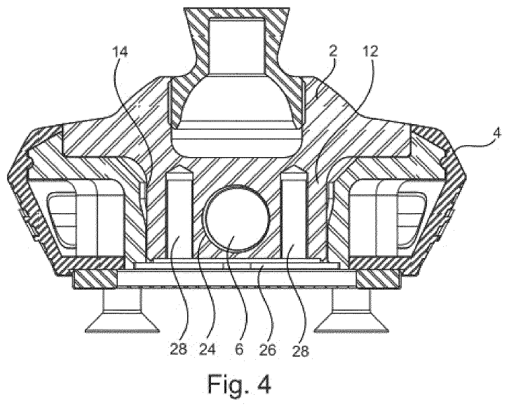

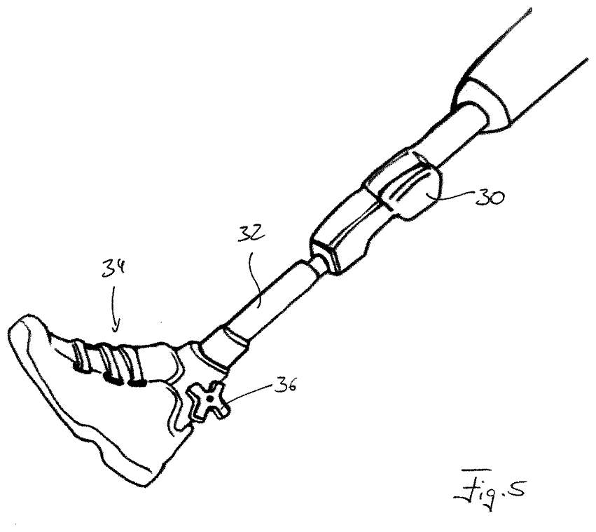

FIG. 4—the connection device in a second sectional view,

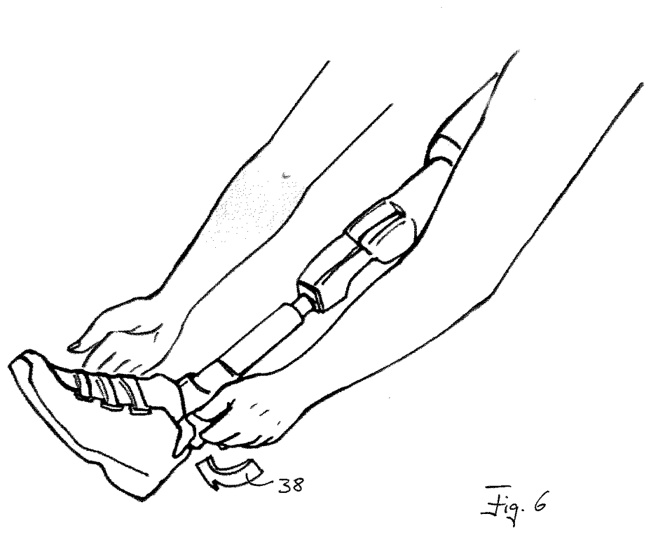

FIGS. 5 to 11—schematic depictions of the changing a prosthetic foot of a prosthesis according to a further example of an embodiment of the present invention, and

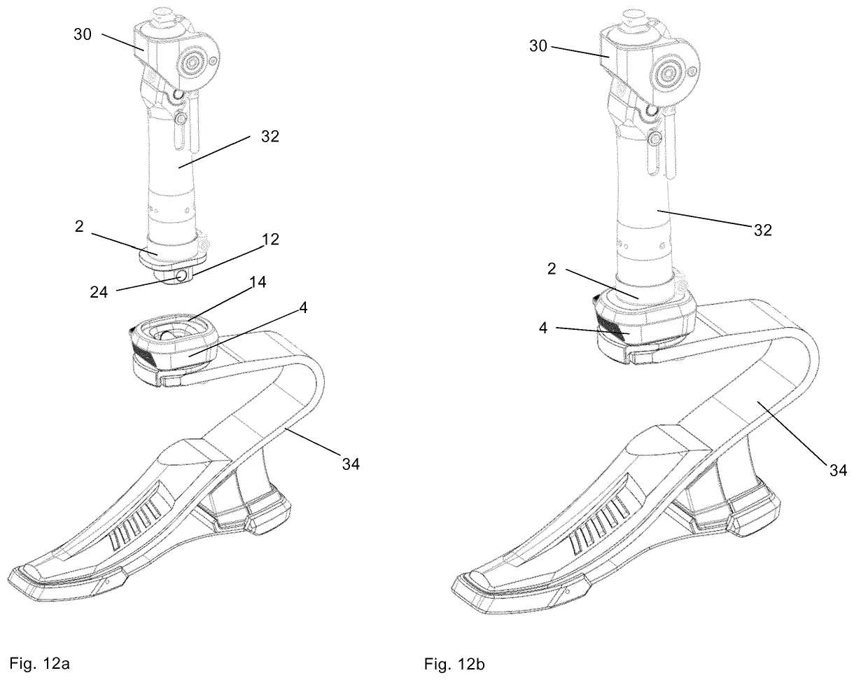

FIGS. 12 to 14—connection devices integrated in prosthesis components.

FIG. 1 depicts a connection device 1 according to a first example of an embodiment of the present invention. It has a first holding element 2, a second holding element 4 and a locking element 6. The first holding element 2 can be connected to a first prosthesis component, not depicted here, via a fixing element; in the example of an embodiment shown, said fixing element is a pyramid adapter 8. Screws 10 are depicted on the second holding element 4 by means of which the second holding element 4 can be fixed to a second prosthesis component, also not depicted here. The first holding element 2 features a projection 12, which can be inserted into a recess 14 provided in the second holding element 4. In this state, bores provided in both the projection 12 and a base body 16 of the second holding element 4 are moved to overlap with one another in such a way that the locking element 6 is inserted into the bores and can be fixed via an outer thread 18, which is designed to correspond to an inner thread provided in one of the bores.

This situation is depicted in FIG. 2. The first holding element 2, which is inserted in the second holding element 4, is clearly recognizable. The locking element 6 has also been inserted into the bores, not depicted here, and screwed in via the thread.

FIG. 3 shows the situation in a sectional view. The outer thread 18 of the locking element 6 engages with an inner thread 20 in a bore 22 in the base body 16 of the second holding element 4. In the example of an embodiment shown, the bore 22 is designed to be conical. This is advantageous but not absolutely necessary. A corresponding bore 24 is situated in the first holding element 2, said bore extending completely through the first holding element 2 in the example of an embodiment shown. The locking element 6 protrudes through the two bores 22, 24, thereby locking together both holding elements 2, 4 and therefore also prosthesis components—not depicted here—arranged on these holding elements 2, 4.

FIG. 4 depicts a sectional view through the situation shown in FIG. 2, rotated by 90° relative to the section in FIG. 3. One is now looking along the direction of longitudinal extension of the locking element 6, which is situated in the bore 24 in the first holding element 2.

In the example of an embodiment shown, the second holding element 4 features a metal plate 26, which is arranged at the base of the recess 14 in which the projection 12 of the first holding element 2 is situated. In the example of an embodiment shown, two permanent magnets 28 are arranged in this projection 12; said magnets exert a holding force on the metal plate 26. Of course, further permanent magnets may also be provided in lieu of the metal plate 26. This renders it possible for the connection device to exert a holding force between the two holding elements 2, 4.

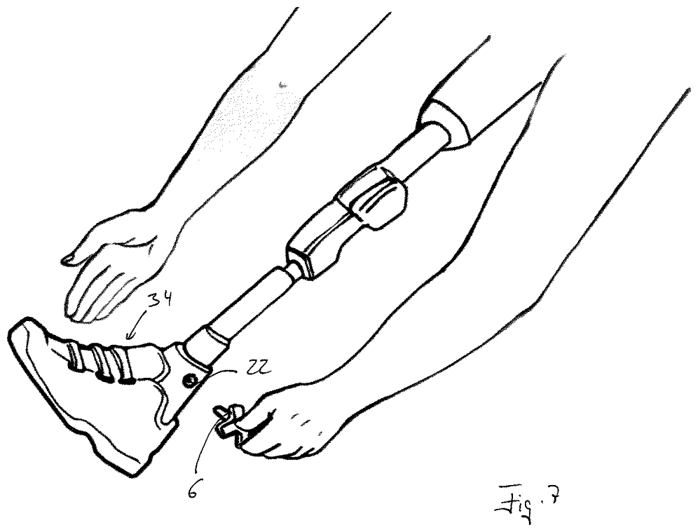

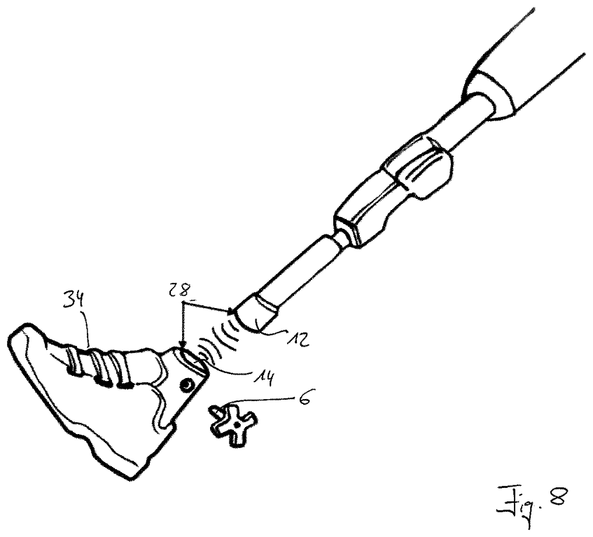

FIG. 5 depicts a prosthesis according to an example of an embodiment of the present invention. It features an artificial knee 30 to which an artificial lower leg 32 is attached; a foot 34 in a shoe is arranged on said artificial lower leg. Another embodiment of the present invention relates to a lower leg prosthesis for patients who still have a natural knee joint. In this case, the following configurations apply analogously and can also be transferred to other configuration examples. The connection device is situated between the foot 34 and the artificial lower leg 32; of said connection device, only an activation element 36 of the locking element 6 is depicted in the view shown. If the foot 34 is now to be swapped, the locking element 6 must first be released along the arrow direction 38 depicted in FIG. 6. By rotating the activation element 36 and thus the locking element 6 in the arrow direction 38, the outer thread 18 is disengaged from the inner thread 20. It is then possible, as shown in FIG. 7, to remove the locking element 6 from the bore 22 and the bore 24, not depicted. It is clear that it is not necessary to hold the foot 34 as a holding force is applied by the two holding elements 2, 4 that form the holding device. As a result, the foot 34 cannot fall off.

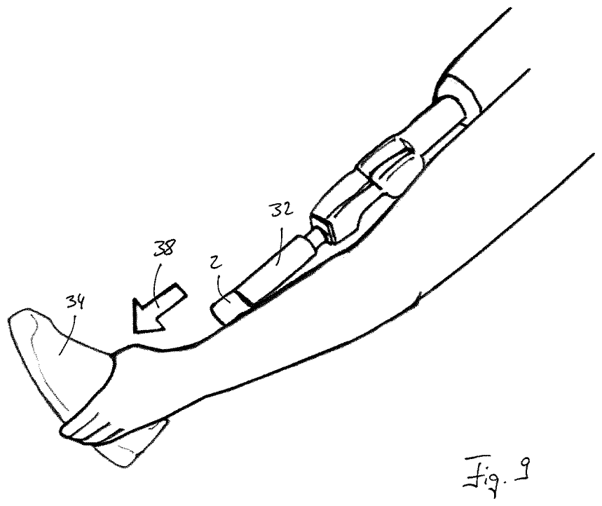

FIG. 8 depicts the released state. Permanent magnets 28 are arranged in the projection 12 of the first holding element 2 and in the recess 14 of the second holding element 4, said permanent magnets exerting an attractive force on one another. This is only schematically indicated. The locking element 6 is still removed from the bores 22, 24.

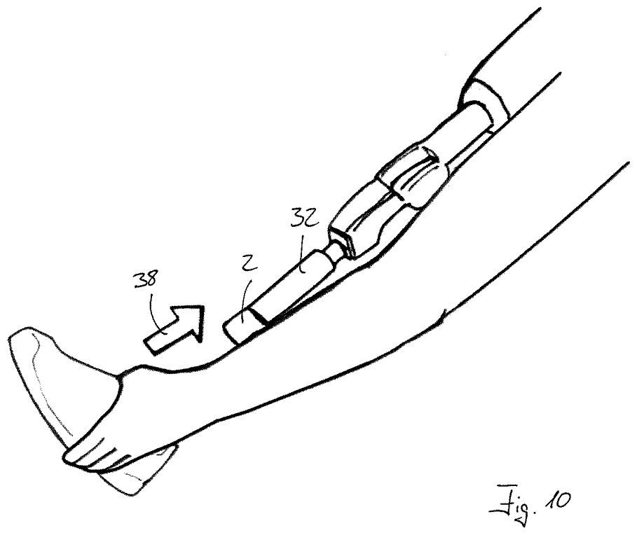

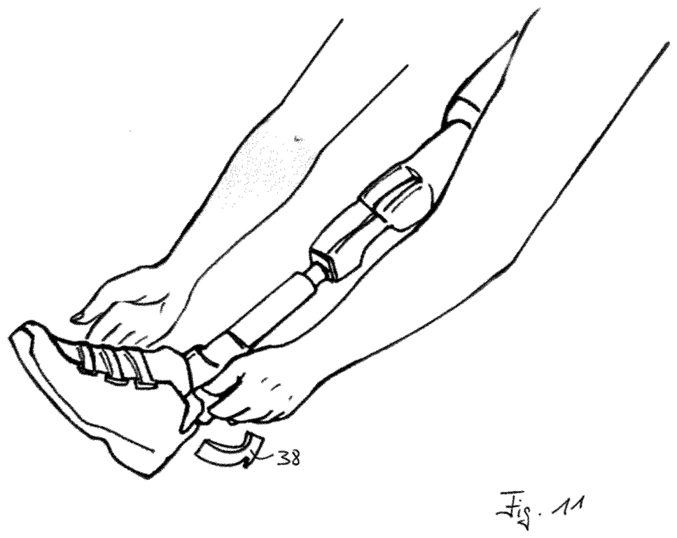

The foot 34 is now removed from the rest of the prosthesis and can be swapped. In FIG. 9, a new foot 34 is already provided; said foot is to be assembled. To this end, the second holding element 4—not depicted here—arranged on said foot is engaged with the first holding element 2, which is situated on the artificial lower leg 32, in such a way that a holding force is exerted by the two holding elements 2,4 that form the holding device. To this end, the first holding element 2 must be moved along the arrow direction 38 in FIG. 9 and the second holding element 4 along the arrow direction 38 in FIG. 10. As shown in FIG. 11, the locking element 6—not depicted—can subsequently be inserted into the bores 22, 24 and locked along the arrow direction 38 in FIG. 11.

FIGS. 12a and 12b depict an artificial lower leg 32 as a first prosthesis component, at the upper end of which an artificial knee 30 is arranged. The first holding element 2 is integrated at the lower end, wherein FIG. 12a depicts the projection 12 in which the bore 24 for the locking element 6—not depicted in FIGS. 12a and 12b—is situated. An artificial foot 34 is depicted as a second prosthesis component, at the upper end of which the second holding element 4 is integrated. The projection 12 of the first holding element 2 is inserted into its recess 14. FIG. 12b shows the configuration in the connected state. In comparison with an embodiment with a conventional artificial lower leg that is connected to a conventional artificial foot, wherein the first holding element 2 and the second holding element 4 are designed as separate components arranged on the respective prosthesis components, this allows up to 80 mm installation height and up to 300 g in weight per prosthesis to be saved.

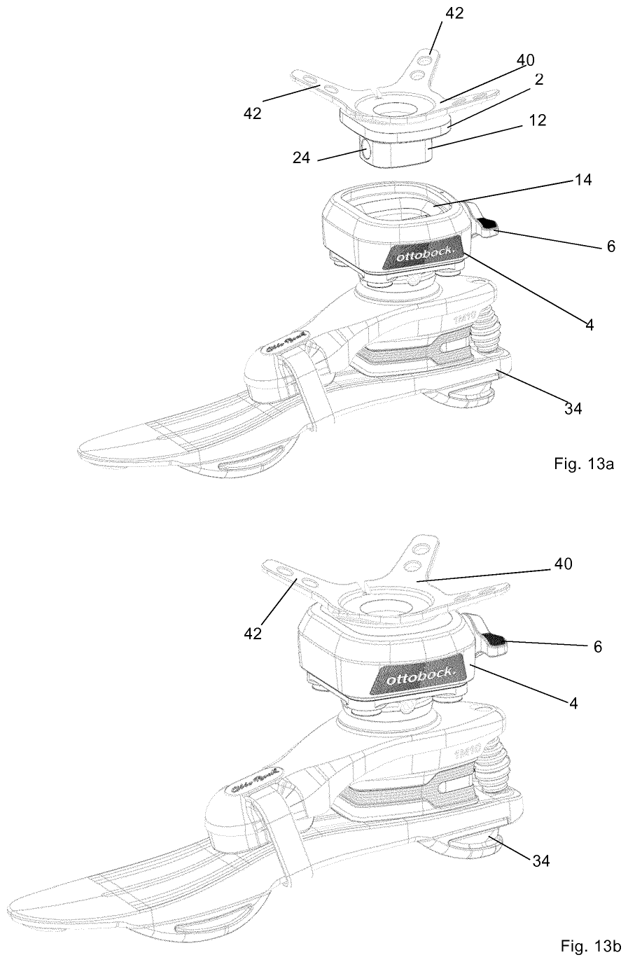

FIGS. 13a and 13b show two further prosthesis components. The artificial foot 34 is depicted, which is to be connected to a so-called casting adapter 40. A casting adapter renders it possible to arrange further components on a prosthesis socket that is yet to be produced, not depicted in FIGS. 13a and 13b. This casting adapter 40 has four arms 42, which can be cast in the material of the prosthesis socket, for example. Casting adapters 40 with different numbers of arms 42 are available. Casting adapters without arms 42 are also used. The projection 12 of the first holding element 2 is again situated on the lower side of the casting adapter 40, wherein the bore 24 for the locking element 6 is depicted in said projection. The second holding element 4 with the recess 14 is situated on the artificial foot 34. The locking element 6 is also shown, by way of which the two holding elements 2, 4 are locked together. FIG. 13a depicts the unconnected state and FIG. 13b the connected state.

With the embodiment depicted in FIGS. 13a and 13b, up to 40 mm installation height and up to 100 g in weight per prosthesis can be saved. For example, if used in a knee joint, 40 mm installation height and up to 200 g in weight can be saved.

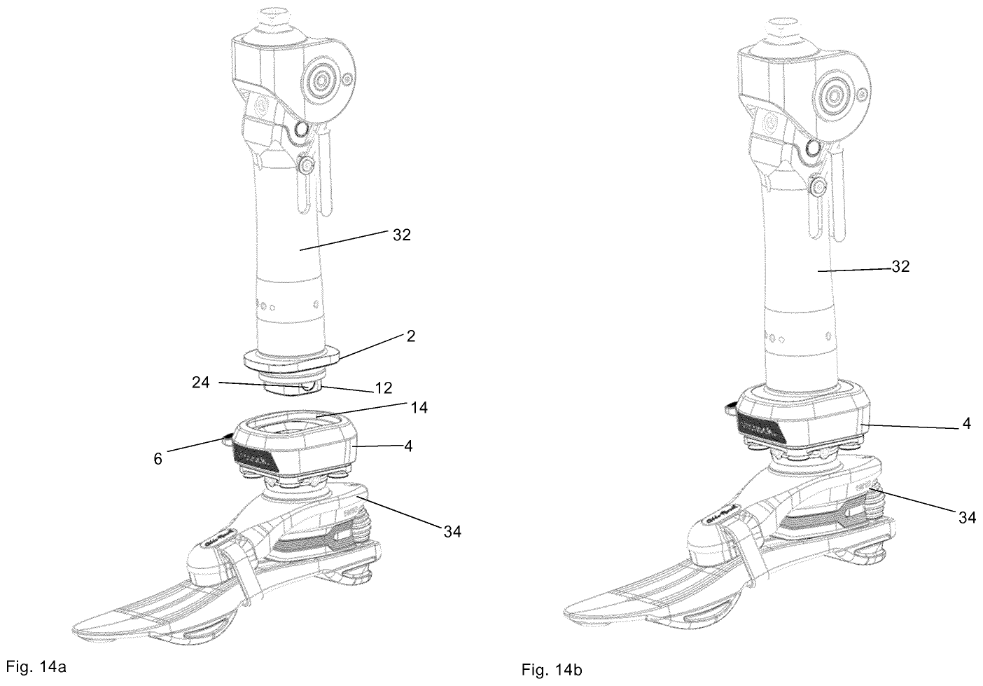

FIGS. 14a to 14b show another embodiment. The depiction corresponds to the depictions in FIGS. 12a and 12b. The first holding element 2 with the projection 12 is arranged on the artificial lower leg 32, while the second holding element 4 with the recess 14 is positioned on the artificial foot 34. The locking element 6 can also be seen, which is guided through the bore 24 depicted in FIG. 14a when in the locked state. With such configurations, a large installation height of 80 to 90 mm can be saved, for example, because a conventional double adapter with a system height of 82 mm alone can then be replaced.

### REFERENCE LIST

2 first holding element
4 second holding element
6 locking element
8 pyramid adapter
10 screw
12 projection
14 recess
16 base body
18 outer thread
20 inner thread
22 bore
24 bore
26 metal plate
28 permanent magnet
30 artificial knee
32 artificial lower leg
34 foot
36 activation element
38 arrow direction
40 casting adapter
42 arm

1. A prosthesis for a lower extremity, the prosthesis comprising:
a first prosthesis component;
a second prosthesis component;
at least one locking element to lock the first prosthesis component relative to the second prosthesis component;
at least one holding device, by way of which the first prosthesis component is detachably held on the second prosthesis component, the at least one holding device having a first holding element arranged on the first prosthesis component, and a second holding element arranged on the second prosthesis component, the first holding element having a projection with a first bore, the second holding element having a base body with a second bore, the at least one locking element engaging the first and second bores to lock the first holding element to the second holding element.

2. (canceled)

3. The prosthesis according to claim 1, wherein the first holding element and the second holding element are positive-locking elements that are designed to correspond to one another.

4. The prosthesis according to claim 1, wherein the first holding element exerts a magnetic holding force on the second holding element.

5. The prosthesis according to claim 4, wherein at least one of the first holding element and the second holding element comprise at least one permanent magnet.

6. The prosthesis according to claim 1, wherein the first holding element is arranged on a first contact element and the second holding element is arranged on a second contact element, wherein the first and second contact elements lie next to one another when the first prosthesis component is locked relative to the second prosthesis component.

7. The prosthesis according to claim 6, wherein the first contact element features a projection and the second contact element features a recess that is designed to correspond to the projection.

8. The prosthesis according to claim 1, wherein the holding device can be released with one hand and without a tool.

9. The prosthesis according to claim 1, wherein at least one of the prosthesis components can only be locked when the at least one holding device is holding the prosthesis components together and the at least one holding device can only be released when the prosthesis components are unlocked.

10. A connection device for a prosthesis according to claim 1, wherein the connection device comprises the at least one holding device and the at least one locking element, the at least one holding device having the first holding element arranged on the first prosthesis component and the second holding element arranged on the second prosthesis component.

11. A prosthesis for a lower extremity, the prosthesis comprising:
a first prosthesis component;
a second prosthesis component;
a locking element to lock the first prosthesis component relative to the second prosthesis component;
a holding device to connect the first prosthesis component to the second prosthesis component, the holding device having a first holding element arranged on the first prosthesis component and having a projection with a first bore, and a second holding element arranged on the second prosthesis component and having a base body with a second bore, the locking element insertable into the first and second bores to secure the first holding element to the second holding element.

12. The prosthesis according to claim 11, wherein the first holding element and the second holding element are positive-locking elements that mate with one another.

13. The prosthesis according to claim 11, wherein the first holding element exerts a magnetic holding force on the second holding element.

14. The prosthesis according to claim 11, wherein at least one of the first holding element and the second holding element comprise a permanent magnet.

15. The prosthesis according to claim 11, wherein the first holding element is arranged on a first contact element and the second holding element is arranged on a second contact element, the first and second contact elements arranged next to each other when the first prosthesis component is secured relative to the second prosthesis component.

16. The prosthesis according to claim 15, wherein the first contact element includes a projection and the second contact element includes a recess that mates with the projection.

17. The prosthesis according to claim 11, wherein the holding device is configured to be released with one hand and without a tool.

18. The prosthesis according to claim 11, wherein at least one of the prosthesis components can only be locked when the holding device is holding the prosthesis components together and the holding device can only be released when the prosthesis components are unlocked.

19. A connection device for a prosthesis according to claim 1, wherein the connection device comprises the holding device and the locking element.
